(19) United States
(12) Patent Application Publication
(10) Pub. No.: US 2010/0266710 A1
(43) Pub. Date: Oct. 21, 2010

(54) PHARMACEUTICAL PREPARATIONS COMPRISING ELECTROCHEMICALLY ACTIVATED HYPOCHLORITE SOLUTIONS

(76) Inventor: Mihran Baronian, Toffen (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/670,513

(22) PCT Filed: Jul. 28, 2008
(86) PCT No.: PCT/EP08/06193

§ 371 Date: Jun. 26, 2010

(30) Foreign Application Priority Data
Jul. 26, 2007 (US) 60952035
Nov. 15, 2007 (US) 60988181

(51) Int. Cl. A61K 33/20; A61K 31/216; A61K 31/728; A61P 27/04; A61P 27/06; A61P 27/02; C11D 3/02

(52) U.S. Cl. 424/661; 514/530; 514/54; 510/112

(57) ABSTRACT

The present application refers to pharmaceutical preparations comprising an active ingredient and a carrier wherein the carrier comprises an aqueous electrochemically activated salt solution.

The present application refers to pharmaceutical preparations comprising an active ingredient and a carrier wherein the carrier comprises an aqueous electrochemically activated salt solution.

Annolyte® or Imecalyte® is a neutral electrochemically activated salt solution, which is a highly effective disinfectant. This activated solution may be obtained by electrolysis of sodium chloride solutions. It can be used in applications like surface disinfection, e.g. of working plates, tables, floors, etc., for cold sterilizing procedures, in agriculture for the elimination of microbial organisms, for wash and laundry applications in swimming pools and even as prophylaxis against athlete's foot. A device for manufacturing neutral electrochemically activated salt solutions is described in EP-A-1 728 768, which is herein incorporated by reference. The Applicant, however, has no knowledge of these solutions as carriers for use in pharmaceutical compositions.

WO 2004/031077 discloses a device for producing a biocidal solution by electrolytic treatment of an aqueous salt solution. The content of this document is herein incorporated by reference.

WO 2005/065388 discloses an oxidative reduction potential water solution and its use as disinfectant or for wound treatment. The content of this document is herein incorporated by reference.

Subject-matter of the present invention is the use of aqueous electrochemically activated salt, particularly hypochlorite salt solutions as carriers in pharmaceutical preparations. The pharmaceutical preparations preferably comprise at least two separate phases, wherein a first phase comprises the active ingredient and a second phase comprises the carrier.

In a first aspect the present invention refers to a pharmaceutical preparation comprising an active ingredient and a carrier, wherein the carrier comprises an aqueous electrochemically activated salt solution having a content of free chlorine between 1 and 500 mg/l and a positive redox potential of between +150 and +1350 mV. The active ingredient is preferably physically separated from the carrier, e.g. the active ingredient is present in a phase separate from the electrochemically activated salt solution. If the active ingredient is, however, sufficiently stable in the presence of the electrochemically activated salt, the pharmaceutical preparation may also consist of a single phase, e.g. an aqueous solution.

In a further aspect the present invention refers to the use of an aqueous electrochemically activated salt having a content of free chlorine between 1 and 500 mg/l and a positive redox potential of between +150 and +1350 mV as a carrier for the manufacture of a pharmaceutical preparation.

The aqueous electrochemically activated solution has a content of free chlorine (as determined by amperometric measurement (DPD) according to U.S. Pat. No. 4,278,507, which is herein incorporated by reference), which provides sufficient activity, e.g. disinfectant or anti-microbial activity, without detrimentally affecting the stability of the preparation. The content of free chlorine may be adjusted by diluting concentrated electrochemically activated salt solutions in a ratio of e.g. 1 part (vol) salt solution to from 1 to 250 parts (vol) of a physiologically acceptable carrier such as water, buffer or a saline solution. Preferably, the content of free chlorine is between 1 and 500 mg/l, particularly between 10 and 400 mg/l and more particularly between 100 and 350 mg/l.

The redox potential of the electrochemically activated salt solution is at least between +150 mV, preferably at least +200 mV, more preferably at least +300 mV, even more preferably at least +400 mV, even more preferably at least +500 mV and upt to +1330 mV, preferably up to +1200 mV. In certain embodiments, the redox potential is +650 and +950 mV, particularly between +700 and +900 mV. The electrochemically activated salt solution is preferably an alkaline metal hypochlorite solution, e.g. lithium, sodium or potassium hypochlorite solution. More preferably, the solution is a sodium hypochlorite solution.

The electrochemically activated salt solution usually has a pH from 2-8. In certain embodiments the pH may be from 2-5, e.g. from 2-4, from 2-3 or from 2-2.8. In other embodiments, the pH may be from 5-8, particularly from 5.9 to 7.6 and more particularly from 6.7 to 7.4.

The content of chlorate and/or the content of chlorite is preferably below toxic levels, e.g. less than 10 mg/l. Further, the solution is preferably free from detectable amounts of radicals such as OH radicals and from ozone. Furthermore, the solution is preferably free from heavy metal ions, e.g. from Mo ions.

The active ingredient can be any medicament suitable for use in human or veterinary medicine, e.g. selected from hydrophilic active ingredients or from lipophilic agents. In certain embodiments, it is preferred that the active ingredient is selected from lipophilic or amphiphilic ingredients, i.e. ingredients, which have a butanol-water distribution coefficient of at least 0.5, preferably of at least 1. In other embodiments, the active ingredient is a hydrophilic ingredient such as polysaccharide. For example, the active agent may be selected from agents for the treatment of glaucoma, e.g. prostaglandines such as Latanoprost, beta-blockers such as Timolol, agents for lowering increased intraocular pressure such as Dorzolamide, agents for the treatment of the dry eye syndrome (ophthalmic lubricants), such as hydroxypropylmethylcellulose (hypromellose) and hyaluronic acid and other pharmaceutically active agents.

The pharmaceutical preparation of the present invention is stabilized against microbial degradation. Thus, the preparation is suitable for multi-use applications. The preparation for multi-use applications may even be devoid of conventional preservatives.

The preparation has a stability against microbial degradation of preferably at least 6 months, more preferably at least 12 months even when stored at room temperature. Preferably, the anti-microbial activity of the electrochemically activated salt solution may be determined by measuring the product c×t of concentration (c) and action time (t) according to a method described by Schleupen, GWF, 1996. Preferably, the value of c×t is 1 mg/l×min or less, more preferably 0.5 mg/l×min or less in order to obtain reduction rates of 10⁶ against microorganisms such as Pseudomonas aeruginosa or Legionella pneumophila.

The preparation may be for any type of administration, e.g. for local or for systemic administration. For example, the preparation is for ocular, nasal, otic, topical, pulmonal, mucosal, oral or intraperitoneal administration, e.g. for administration by injection. Preferred preparations are for ocular administration, e.g. for the treatment of glaucoma or of the dry eye syndrome.

In one preferred embodiment of the invention, the pharmaceutical preparation comprises a single phase comprising both the active ingredient and the electrochemically activated salt. In this embodiment, the active ingredient is sufficiently stable against degradation in the presence of the chemically activated salt. Suitable examples of active ingredients are polymers selected from polysaccharides and polyvinylpyrrolidone polymers. The polysaccharides may e.g. be selected from cellulose or cellulose derivatives or glucosamino glycanes such as heparin, heparane sulfate and hyaluronic acid. More preferably, the preparation may comprise an ophthalmic lubricant and a carrier, wherein the carrier comprises an aqueous electrochemically activated salt solution having a content of free chlorine as described above, i.e. between 1 and 500 mg/l and a redox potential as described above, i.e. between +150 and +1350 mV. The ophthalmic lubricant may be a polymer, e.g. polysaccharide or a polyvinyl-pyrrolidone, for example cellulose or a cellulose derivative such as hydroxypropylmethylcellulose or a glucosamine glycane such as hyaluronic acid. The pharmaceutical preparation is preferably a homogenous aqueous solution. In this embodiment it is preferred that no further active agents and/or no preservatives are present in the preparation. The preparation according to said embodiment can be used for treating and/or preventing the dry eye syndrome.

In another embodiment, the preparation preferably forms at least two separate phases wherein the active agent is present in a first phase and the electrochemically activated salt solution is present in a second phase separate from the first phase. The first phase may be a solid particulate phase, a liquid hydrophobic phase or a solid or liquid phase having a barrier towards the second phase which is an aqueous phase comprising the carrier. Thus, the preparation may be an emulsion, e.g. a microemulsion, or a liposomal preparation, or a microcapsule preparation, or dispersion wherein the active agent may be emulgated or dispersed optionally in the presence of a carrier, e.g. a lipophilic carrier and/or surfactants, within the aqueous carrier. The active agent may thereby be physically separated from the electrochemically activated salt solution.

Especially preferred are microemulsions as described in EP 07 008 347.1, which is herein incorporated by reference.

In addition to the active agent and the electrochemically activated salt solution, the preparation may contain other known ingredients, e.g. buffers, adjuvants, auxiliary agents, fillers, diluents, etc.

The preparation of the invention may be used in human or veterinary medicine.

Still a further aspect of the present invention refers to the use of an electrochemically activated salt solution as described above for the cleaning of contact lenses, e.g. glass or plastic contact lenses. In this embodiment, the solution may be devoid of any active agent or may comprise an active agent, e.g. a polymer as described above.

Still a further aspect of the present invention refers to the use of an electrochemically activated salt solution as described above for the rinsing of body cavities, e.g. as a solution for the nasal, ocular or otic application. In this embodiment, the solution may be devoid of any active agent, or may comprise an active agent, e.g. a polymer as described above.

Further, the present invention shall be described in more detail by the following examples.

### EXAMPLE 1

### Preparation and Characterisation of Microemulsions with an Electrochemically Activated Salt Solution as Carrier

### 1.1 Composition of Tested Formulations

The following microemulsions comprising 0.0050% latanoprost as an active ingredient were prepared. All percentages refer to weight percent.

| | ME1 | ME2 | ME3 | ME4 |
|---|---|---|---|---|
| Latanoprost [%] | 0.0050 | 0.0050 | 0.0050 | 0.0050 |
| Ethyl oleate [%] | 4.38 | 4.38 | 4.01 | 3.02 |
| Tween 80 [%] | 4.52 | 4.53 | 4.01 | 3.02 |
| Tween 20 [%] | 2.21 | 2.21 | 2.01 | 1.46 |
| Water [%] | 75.90 | 85.55 | 66.96 | 50.01 |
| Sodium Hyaluronate [%] | 0.025 | 0.025 | 0.020 | 0.020 |
| Sodium Citrate [%] | 0.90 | 0.89 | 0.76 | 0.58 |
| Sorbitol [%] | 1.92 | 1.82 | 1.61 | 1.18 |
| Calcium Citrate [%] | 0.010 | 0.010 | 0.0041 | 0.0032 |
| IMECALYTE ® [%] | 10.11 | 0.56 | 20.61 | 40.7 |
| Redox Potential [mV] | 244 | 188 | 325 | 398 |

### 1.2 Stability of Latanoprost in IMECALYTE® Microemulsions

The stability of latanoprost in microemulsions comprising an electrochemically activated salt solution (IMECALYTE®) as a carrier was determined by HPLC. As comparison, the stability of latanoprost in IMECALYTE® solutions was tested.

The amount of latanoprost (μg/ml) after storage at room temperature for the indicated time periods was as follows:

| | ME1 | ME2 | Pure solution | Pure solution |
|---|---|---|---|---|
| Room | 10% | 0.5% | 0.5% | 10% |
| temperature | IMECALYTE ® | IMECALYTE ® | IMECALYTE ® | IMECALYTE ® |
| t = 0 | 60.3 | 60.2 | 58.8 | 58.8 |
| t = 5 min | | | | 2.1 |
| t = 30 min | | | | n.d. |
| t = 2 weeks | 60.2 | 60.3 | 48.2 | — |
| t = 4 weeks | 60.4 | 60.3 | 35.4 | — |
| t = 12 weeks | 59.6 | 60.2 | 17.4 | — |

The results show that latanoprost is protected from degradation induced by IMECALYTE® in microemulsion formulations ME1 and ME2. In contrast thereto, latanoprost is unstable in IMECALYTE® solutions. In 0.5% IMECALYTE®, significant degradation is detected after two weeks. In a 10% IMECALYTE® solution, latanoprost is completely degraded within a few minutes.

### 1.3 Antimicrobial Activity

The antimicrobial growth activity of latanoprost containing microemulsions ME1-4 against Staphylococcus aureus and Pseudomonas aeruginosa was tested. The starting concentration of S. aureus was 2.82×10⁵/ml for ME1 and ME2 and 1.7×10⁶/ml for ME3 and ME4. The starting concentration of P. aeruginosa was 2.15×10⁶/ml for ME1 and ME2, 3.18×10⁵/ml for ME3 and 3.18×10⁶/ml for ME4.

After 14 days, no viable S. aureus and P. aeruginosa microorganisms (concentration <10/ml) could be detected in ME1, ME3 and ME4. In ME2, after 28 days, no S. aureus organisms were detectable. Growth of P. aeruginosa, however, could not be significantly inhibited in ME1.

The result of Example 1 shows that an active ingredient such as latanoprost is protected from degradation in IMECALYTE®-based microemulsions. Further, these microemulsions exhibit significant antibacterial properties.

### EXAMPLE 2

### Preparation and Characterisation of IMECALYTE®-Based Hyaluronic Acid Formulations

The following hyaluronic acid formulations were prepared. All percentages are weight percentages.

| | HS1 | HS2 |
|---|---|---|
| Sodium Hyaluronate [%] | 0.30 | 0.30 |
| Sodium citrate [%] | 0.85 | 0.96 |
| Sorbitol [%] | 1.78 | 1.99 |
| Water for inject. [%] | 86.90 | 96.25 |
| IMECALYTE ® [%] | 10.17 | 0.50 |
| Redox Potential [mV] | 367 | 181 |

It was tested if IMECALYTE® has a relevant effect on formulation properties such as drop erogation. The contact angle of erogated drops on a reference paper surface was measured immediately after manufacture of formulations HS1 and HS2 and after storage for three months at room temperature. As comparison, a hyaluronic acid formulation corresponding to HS2 (however without IMECALYTE®) was used.

The following results were obtained:

| | | Contact angle t = 3 |
|---|---|---|
| | Contact angle t = 0 | months |
| HS2 no IMECALYTE ® | 120.7° +/− 6.3 | 121.5° +/− 4.8 |
| HS2 | 118.8° +/− 3.3 | 119.6° +/− 5.2 |
| HS1 | 116..9° +/− 5.6 | 118.4° +/− 5.6 |

Further, the spreading of erogated drops of different hyaluronic acid formulations immediately after manufacture (t=0) and after storage for three months at room temperature (t=3 months) was determined.

| | DROP DIAMETER 2R mm | | |
|---|---|---|---|
| | t = 5 sec | t = 30 sec | t = 300 sec |
| at t = 0 | | | |
| HS2 no IMECAL. | 1.8 | 1.7 | 2.0 |
| HS2 | 1.4 | 1.5 | 1.5 |
| HS1 | 1.5 | 1.4 | 1.4 |
| at t = 3 | | | |
| months at RT | | | |
| HS2 no IMECAL. | 1.9 | 2.0 | 1.9 |
| HS2 | 1.5 | 1.4 | 1.5 |
| HS1 | 1.6 | 1.5 | 1.5 |

The above results show that IMECALYTE® has no relevant effect on the drop erogation characteristics of hyaluronic acid formulations. The quality of erogation and drop formation of the IMECALYTE®-based solutions was very good.

Further, the antimicrobial activity of IMECALYTE®-based hyaluronic acid formulations HS1 and HS2 was determined. As test organisms, S. aureus (starting concentration 7.27×10⁵/ml) and P. aeruginosa (starting concentration 1.29×10⁶/ml) were used. With solution HS1, the concentration of viable P. aeruginosa was below 10²/ml after 6 h. With S. aureus, no viable microbes could be determined after 14 days. With the formulation HS2, viable P. aeruginosa organisms could not be detected after 7 days. Viable S. aureus organisms could not be detected after 14 days.

The above results show that IMECALYTE®-based hyaluronic acid formulations are both stable and antimicrobially active.

1. Pharmaceutical preparation comprising an active agent and a carrier, wherein the carrier comprises an aqueous electrochemically activated salt solution having a content of free chlorine between 1 and 500 mg/l and a redox potential of between +150 and +1350 mV, wherein the active agent is present in a phase separate from the electrochemically activated salt solution.

2. The preparation of claim 1, wherein the content of free chlorine is between 10 and 400 mg/l.

3. The preparation of claim 1, wherein the redox potential is between +650 and +950 mV.

4. The preparation according to claim 1, wherein the electrochemically activated salt solution is a sodium hypochlorite solution.

5. The preparation according to claim 1, wherein the hypochlorite solution has a pH from 2 to 8.

6. The preparation according to claim 1, wherein the content of chlorate and/or the content of chlorite is less than 10 mg/l.

7. The preparation according to claim 1, wherein the active agent is selected from prostaglandines, beta-blockers, agents for lowering increased intraocular pressure, or ophthalmic lubricants.

8. The preparation according to claim 1, which is for multi-use application.

9. The preparation according to claim 1, which is for local or for systemic administration.

10. The preparation according to claim 1, which is for ocular, nasal, otic, topical, pulmonal, mucosal, oral or intraperitoneal administration.

11. The preparation according to claim 1, wherein the active agent is present in a solid particulate phase, a liquid hydrophobic phase or a solid or liquid phase having a barrier towards the electrochemically activated salt solution.

12. The preparation according to claim 1, which is an emulsion or dispersion.

13. A method of preparing a pharmaceutical composition comprising combining an active ingredient with a pharmaceutically acceptable carrier, wherein said carrier comprises an aqueous electrochemically activated salt solution having a content of free chlorine between 1 and 500 mg/l and a redox potential of between +150 and +1350 mV.

14. (canceled)

15. (canceled)

16. A method of cleaning of contact lenses comprising applying to said contact lenses an aqueous electrochemically activated salt solution having a content of free chlorine between 1 and 500 mg/l and a redox potential of between +150 and +1350 mV.

17. A method of rinsing of body cavities for nasal, ocular or otic application, comprising rinsing a body cavity with an aqueous electrochemically activated salt solution having a content of free chlorine between 1 and 500 mg/l and a redox potential of between +150 and +1350 mV.

18. (canceled)

19. Pharmaceutical preparation comprising an ophthalmic lubricant and a carrier wherein the carrier comprises an aqueous electrochemically activated salt solution having a content of free chlorine between 1 and 500 mg/l and a redox potential of between +150 and +1350 mV.

20. The preparation of claim 19, wherein the preparation is an aqueous solution.

21. The preparation of claim 19, wherein the ophthalmic lubricant is a polysaccharide or polyvinyl pyrrolidone polymer, a cellulose derivative, or a glucosamine glycane.

22. The preparation as claimed in claim 21, wherein the cellulose derivative is hydroxypropylmethylcellulose and the glucosamine glycane is hyaluronic acid.

23. The preparation according to claim 19, wherein no further active agents and/or preservatives are present.

24. A method of treating and/or preventing the dry eye syndrome comprising applying a pharmaceutical preparation according to claim 19.

25. Pharmaceutical preparation comprising an active ingredient and a carrier, wherein the active ingredient is a polysaccharide or a polyvinylpyrrolidone polymer, a cellulose derivative, or a glucosamine glycane.
